(19) United States
(12) Patent Application Publication
(10) Pub. No.: US 2009/0060996 A1
(43) Pub. Date: Mar. 5, 2009

(54) Formulations of Clopidogrel Bisulphate

(76) Inventor: Torfi E. Kristjansson, Reykjavik (IS)

Correspondence Address: WENDEROTH, LIND & PONACK, L.L.P., 2033 K STREET N. W., SUITE 800, WASHINGTON, DC, 20006-1021, US

(21) Appl. No.: 12/223,693

(22) PCT Filed: Feb. 9, 2007
(86) PCT No.: PCT/IS2007/000006

§ 371 Date: Sep. 29, 2008

(30) Foreign Application Priority Data
Feb. 10, 2006 (IS) 8294

(51) Int. Cl. A61K 9/20; A61K 31/44; A61P 43/00

(52) U.S. Cl. 424/465; 514/301

(57) ABSTRACT

This invention relates to pharmaceutical tablet formulations of clopidogrel bisulphate which include glyceryl dibehenate as lubricant. The tablets are found to be very stable and to exhibit suitable dissolution characteristics.

### FIELD OF THE INVENTION

The present invention relates to improved tablet formulations of clopidogrel bisulphate.

### TECHNICAL BACKGROUND AND PRIOR ART

Clopidogrel bisulphate, methyl (+)-(S)-a-(2-chlorophenyl)-6,7-dihydrothieno [3,2-c]pyridine-5(4H)-acetate sulphate (1:1), is an inhibitor of ADP-induced platelet aggregation acting by direct inhibition of adenosine diphosphate (ADP) binding to its receptor and of the subsequent ADP-mediated activation of the glycoprotein GPIIb/IIIa complex.

The preparation of useful formulations of clopidogrel bisulphate is complicated due to stability issues.

EP 1 310 245 B1 discloses a pharmaceutical tablet formulation comprising clopidogrel bisulphate and a lubricant selected form the group consisting of zinc stearate, sodium stearyl fumarate and stearic acid. These tablets comprise methylcellulose as filler/binder. The document states that the tablets should preferably exclude microcrystalline cellulose.

WO 00/01364 claims a stable oral pharmaceutical composition which is free or substantially free of magnesium stearate, a water-soluble polyvinyl pyrrolidone and sodium starch glycollate, which comprises a thieno [3,2-c] pyridine derivative and a water-soluble hydrophilic lubricant comprising a polyethylene glycol having an average molecular weight from about 1000 Da to about 30,000 Da.

WO 2005/070464 describes a tablet formulation of clopidogrel bisulphate where magnesium stearate is not used as lubricant. Instead hydrogenated vegetable oil is employed as lubricant.

### SUMMARY OF INVENTION

The object of the present invention is to provide stable clopidogrel bisulphate tablets that preferably are bioequivalent to already marketed clopidogrel tablets.

It has now been found that clopidogrel bisulphate tablets comprising glyceryl dibehenate as lubricant have very good stability and dissolution characteristics.

None of the above references teach or suggest the use of glyceryl dibehenate with clopidogrel bisulphate.

### DETAILED DESCRIPTION

The invention provides a pharmaceutical formulation comprising clopidogrel bisulphate which is depicted by Formula I, and the lubricant glyceryl dibehenate and optionally a disintegrant.

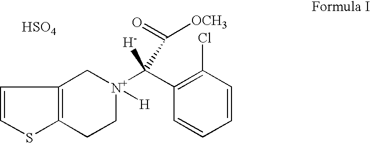

Glyceryl dibehenate may be obtained by esterification of glycerol with behenic acid (C 22:0 fatty acid). The product is provided commercially by Gattefosse s.a. under the trade name Compritol 888 ATO. Compritol has a fatty acid composition with over 83% behenic acid, 40-60% of the fatty acids are in diester form (diglycerides), and 21-35% are in triester form (triglycerides). Accordingly, useful embodiments of the invention comprise a lubricant formulation in the above amount comprising in the range of about 50-100 wt % of glyceryl di- and tribehenate, such as in the range of about 55-95 wt %, including in the range of about 70-80 wt %. Such lubricant formulations would generally contain glyceryl dibehenate and glyceryl tribehenate in a ratio ranging from about 1:1 to about 3:1. Glyceryl dibehenate is a neutral and non-metal containing compound.

Useful embodiments of the invention comprise glyceryl dibehenate as a lubricant in the formulation in an amount in the range of 1-5 wt %, preferably in the range of 2-4 wt % including about 2 wt % and about 2,5 wt % and most preferably 3 wt %.

The pharmaceutical formulations of the present invention typically comprise clopidogrel bisulphate, 1-5 wt % of the glyceryl dibehenate and optionally 1-15 wt % of a disintegrant.

The formulation of the present invention typically further comprises conventional excipients such as a filler, a disintegrant, and a binder. In one embodiment the formulation comprises in the range of 30 to 85% of a filler substance, which may be one of numerous substances generally known in the art, such as e.g. a saccharide (e.g. lactose, sucrose, glucose dextrose, sorbitol or mannitol), dextrates, dextrins, pregelatinized starch, wheat starch, corn starch, or any mixture thereof; preferably microcrystalline cellulose or a mixture of microcrystalline cellulose and any of the above.

It was discovered that contrary to what has been held in the prior art (see esp. EP 1 310 245 B1) microcrystalline cellulose, in the formulations of the present invention, does not have a retarding effect on disintegration and dissolution of the active Ingredient.

The formulation may further comprise in the range of 0.5 to 5 wt % of a disintegrant such as crosslinked sodium carboxymethylcellulose (NaCMC), starch, hydroxypropylcellulose and polyvinyl pyrrolidone (crospovidone). Preferably the disintegrant Is crospovidone. Most preferably the crospovidone is Polyplasdone XL (trade name).

The formulation may additionally comprise other commonly used excipients that are compatible with the active ingredient such as pigments, colorants, sweeteners, taste-masking agents and the like.

The tablets of the invention can be suitably made by direct compression or wet compression, where direct compression is presently preferred. The tablets are preferably film-coated. They are optionally packed into blister, preferably aluminium-aluminium blister in order to achieve Improved shelf life.

A preferred embodiment of a pharmaceutical formulation of the invention includes lactose anhydrous as a filler material, cellulose microcrystalline as a filler/stabilizer and talc. The talc acts as a glidant and has additionally some lubricating characteristics that support the lubricating role of the glyceryl dibehenate.

As shown in the below Examples, tablet formulations according to the invention have superior stability to comparable prior art tablets which results in extended shelf life. A further benefit is that the composition can be adjusted in order to obtain suitable disintegration times, e.g. very short disintegrations times such as in the range of about 3-6 minutes, e.g. about 4, 4.5, 5 or 5.5 minutes, when tested with a standard disintegration test (in accordance with Ph. Eur.; 37° C. in water).

The tablets are preferably coated, conventional coatings which are well known to the skilled person may be employed. In useful embodiments of the invention, the tablets are coated, e.g. by sugar coating or more preferably by film coating. A number of substances may be used for film coating the tablets of the Invention, including methyl cellulose, ethyl cellulose and hydroxymethyl cellulose based coatings as well as methyl hydroxyl ethyl cellulose, hydroxypropyl cellulose and hydroxypropyl methyl cellulose (e.g. Methocel (Dow)) based coatings, coatings based on polymers of methacrylic acids and its esters (e.g. Eudragit systems (Pharm Rohma) and polyvinyl alcohol (PVA) based coatings such as Opadry system. Such coatings allow distinctive coloring and may enhance the stability of the tablets.

The present invention will be further illustrated by means of the following examples. It is however to be understood that the invention is not meant to be limited to the following examples.

### EXAMPLES

### Example 1

### Tablet Formulation

The following materials were combined and direct compression employed to produce 75 mg clopidogrel tablets (97.873 mg clopidogrel bisulphate):

TABLE 1

| | |
|---|---|
| Clopidogrel bisulphate | 35.6 wt % |
| Lactose anhydrous | 31.4 wt % |
| Cellulose microcrystalline | 30.0 wt % |
| Glyceryl dibehenate | 3.0 wt % |

The disintegration time was too long and additionally the stability study revealed unsatisfactory dissolution.

### Example 2

### Tablet Formulation

The following materials were combined and direct compression employed to produce 75 mg clopidogrel tablets (97.873 mg clopidogrel bisulphate):

TABLE 2

| | |
|---|---|
| Clopidogrel bisulphate | 35.6 wt % |
| Lactose anhydrous | 26.4 wt % |
| Cellulose microcrystalline | 25.0 wt % |
| Glyceryl dibehenate | 3.0 wt % |
| Pregelatinised starch | 10.0 wt % |

The disintegration time was rather long for this formulation, as shown in Example 6.

### Example 3

### Tablet Formulation

The following materials were combined and direct compression employed to produce 75 mg clopidogrel tablets (97.873 mg clopidogrel bisulphate):

TABLE 3

| | | |
|---|---|---|
| Clopidogrel bisulphate | 35.6 | wt % |
| Lactose anhydrous | 23.4 | wt % |
| Cellulose microcrystalline | 30.0 | wt % |
| Glyceryl dibehenate | 3.0 | wt % |
| Pregelatinised starch | 5.0 | wt % |
| Talc | 3.0 | wt % |

The disintegration time was shorter that in Example 2 but the stability study revealed that employing the starch lead to a dissolution failure.

### Example 4

### Tablet Formulation

The following materials were combined and direct compression employed to produce 75 mg clopidogrel tablets (97.873 mg clopidogrel bisulphate):

TABLE 4

| | | |
|---|---|---|
| Clopidogrel bisulphate | 35.6 | wt % |
| Lactose anhydrous | 28.4 | wt % |
| Cellulose microcrystalline | 25.0 | wt % |
| Glyceryl dibehenate | 3.0 | wt % |
| Hydroxypropyl cellulose | 5.0 | wt % |
| Talc | 3.0 | wt % |

The results were good but the disintegration time should preferably be shorter.

### Example 5

### Tablet Formulation

The following materials were combined and direct compression employed to produce 75 mg clopidogrel tablets (97.873 mg clopidogrel bisulphate):

TABLE 5

| | | |
|---|---|---|
| Clopidogrel bisulphate | 35.6 | wt % |
| Lactose anhydrous | 28.4 | wt % |
| Cellulose microcrystalline | 25.0 | wt % |
| Glyceryl dibehenate | 3.0 | wt % |
| Crospovidone | 5.0 | wt % |
| Talc | 3.0 | wt % |

The tablets were coated with polyvinyl alcohol (PVA) coating using the Opadry II High Performance system.

The disintegration time proved to be acceptable and fit the desired criteria.

### Example 6

### Disintegration Time

Disintegration time of clopidogrel tablet formulations from Examples 1 to 5:

TABLE 6

| Clopidogrel bisulphate | Disintegration time |
|---|---|
| formulations | [min:sec] |
| Example 1 | 6:10 |
| Example 2 | 7:13 |
| Example 3 | 4:10 |
| Example 4 | 5:07 |
| Example 5 | 3:04 |

The formulation from Example 5 gave the best results with respect to comparable bioequivalence to the marketed product and the shortest disintegration time.

### Example 7

### Stability Test

The table shows results of a stability study at 40° C. and 75% relative humidity for the tablet formulation of Example 5 for 0, 3 and 6 months with regard to impurity B (a-(2-chloropheny)-6,7-dihydrothieno-[3,2-c]pyridine-5-(4H)acetic acid, conversion into the other isomer and total impurities.

TABLE 7

| Months | Impurity B (%) | Isomer (%) | Total impurities (%) |
|---|---|---|---|
| 0 | 0.02 | 0.03 | 0.30 |
| 3 | 0.05 | 0.08 | 0.43 |
| 6 | 0.13 | 0.13 | 0.50 |

This result is excellent and shows that the pharmaceutical formulation is very stable.

### Example 8

### Comparative Stability Test

The stability of tablets produced as described in Example 5 was tested and compared with commercially available Clopidogrel tablets (Plavix® 75 mg) at 40° C. and 75% relative humidity. All values are percentages based on “area under the curve” chromatographic measurements. ‘AI’ means active ingredient (clopidogrel bisulphate).

Two batches were tested indicated as 5a and 5b, against Plavix® tablets (‘P’) with the same dose (75 mg). The results show surprisingly good stability of the tablets of the invention, which compare very favourable to the marketed tablets.

TABLE 8

| | Time [months] | | | | | | | | | | | |
|---|---|---|---|---|---|---|---|---|---|---|---|---|
| | 0 | | | 1 | | | 3 | | | 6 | | |
| Formulation: | 5a | 5b | P | 5a | 5b | P | 5a | 5b | P | 5a | 5b | P |
| meas. amount of AI | 99.2 | 99.3 | 99.0 | 99.1 | 98.9 | 100.0 | 99.1 | 98.5 | 97.2 | 97.6 | 97.8 | 94.0 |
| Impurity A (USP) | 0.02 | 0.02 | 0.04 | 0.04 | 0.03 | 0.11 | 0.05 | 0.05 | 0.67 | 0.17 | 0.14 | 3.53 |
| Isomer | 0.04 | 0.02 | 0.30 | 0.07 | 0.06 | 0.45 | 0.08 | 0.08 | 0.47 | 0.17 | 0.14 | 1.74 |
| Unknown 1 | | | | | | | | | | 0.11 | 0:08 | 0.29 |
| Unknown 2 | | | | | | | | | | 0.20 | 0.20 | 0.11 |
| Total impurities | | | | | | | | | | 0.45 | 0.56 | 5.67 |

1. A pharmaceutical formulation comprising clopidogrel bisulphate and the lubricant glyceryl dibehenate.

2. The formulation of claim 1, where said formulation additionally comprises a disintegrant selected from the group consisting of starch, hydroxypropyl cellulose and crospovidone.

3. The formulation of claim 2, comprising as a disintegrant crospovidone.

4. The formulation of claim 3, wherein said crospovidone is Polyplasdone XL®.

5. The formulation of claim 1 comprising microcrystalline cellulose.

6. The formulation of claim 5 comprising in the range of 10 to 75 wt % microcrystalline cellulose.

7. The formulation of claim 1 comprising lactose.

8. The formulation of claim 7 comprising in the range of 10-75 wt % lactose anhydrous.

9. The formulation of claim 1 comprising microcrystalline cellulose and lactose.

10. The formulation of claim 9 comprising in the range of 10-65 wt % microcrystalline cellulose and in the range of 10-65 wt % lactose anhydrous.

11. The formulation of claim 10 comprising in the range of 15-40 wt % microcrystalline cellulose and in the range of 15-40 wt % lactose anhydrous.

12. The formulation of claim 1 comprising in the range of 1-5 wt % glyceryl dibehenate.

13. The formulation of claim 1 comprising in the range of 1-5 wt % of a lubricant formulation comprising in the range of about 50-100 wt % glyceryl di- and tribehenate.

14. The formulation of claim 1 formulated in tablet dosage form.

15. The formulation of claim 14, wherein said tablets are coated.

16. The formulation of claim 14 wherein said tablets are prepared by direct compression.

17. The formulation of claim 1 which comprises clopidogrel bisulphate, glyceryl dibehenate, crospovidone, lactose anhydrous, cellulose microcrystalline and talc.

18. The formulation of claim 15, wherein said tablets are prepared by direct compression.
